(19) United States
(12) Patent Application Publication
(10) Pub. No.: US 2005/0053656 A1
(43) Pub. Date: Mar. 10, 2005

(54) Compositions and methods for treating pain

(76) Inventor: Jeffrey H. Ping, Cumming, GA (US)

Correspondence Address: SUTHERLAND ASBILL & BRENNAN LLP, 999 PEACHTREE STREET, N.E., ATLANTA, GA, 30309, US

(21) Appl. No.: 10/932,575

(22) Filed: Sep. 2, 2004

Related U.S. Application Data
(60) Provisional application No. 60/500,893, filed on Sep. 5, 2003.

(51) Int. Cl. A61K 31/7024; A61K 31/485

(52) U.S. Cl. 424/464; 514/23; 514/282

(57) ABSTRACT

Compositions comprising of one or more analgesic tannates such as but not limited to hydrocodone tannate, codeine tannate, oxycodone tannate, oxymorphone tannate, morphine tannate, and hydromorphone tannate, alone or in combination with one or more additional active ingredients which are effective when administered for the treatment of pain.

### CROSS REFERENCE TO RELATED APPLICATIONS

The present invention claims the priority benefit of U.S. Provisional Patent Application Ser. No. 60/500,893 filed Sep. 5, 2003, the entire contents of which are hereby incorporated by reference.

### FIELD OF INVENTION

The invention relates to novel methods and compositions for the treatment of pain.

### BACKGROUND OF INVENTION

Analgesics are agents which relieve pain by acting centrally to elevate pain threshold without disturbing consciousness or altering other sensory modalities. The point at which pain is perceived is referred to as the “pain threshold.” If this threshold is raised, more stimuli are required before pain is experienced. The mechanism of action by which analgesic drugs obtund pain (raise the pain threshold) was explained following the discovery of opiate receptors in selected portions of the central nervous system and the subsequent identification of an endogenous substance (enkephalin, one of a group of substances known as endorphins) from the brain with properties similar to morphine.

Analgesics from the opiate, nonopiate addicting and nonopiate, nonaddicting classes are widely known and widely used in the treatment of various types of pain. The opium group of narcotic drugs are among the most powerfully acting and clinically useful drugs producing depression of the central nervous system. Drugs of this group are used principally as analgesics and include well-known compounds such as opium, morphine, morphine sulfate, morphine hydrochloride, codeine, codeine phosphate, codeine sulfate, hydrocodone, hydromorphone, hydromorphone hydrochloride, hydromorphone sulfate, diacetylmorphine, diacetylmorphine hydrochloride, levorphanol tartrate, oxymorphone hydrochloride, and oxycodone hydrochloride.

Hydrocodone is a semisynthetic opioid antitussive and analgesic with multiple actions qualitatively similar to those of codeine. Most of these involve the central nervous system and smooth muscle. The precise mechanism of action of hydrocodone and other opiates is not known, although it is believed to relate to the existence of opiate receptors in the central nervous system. Hydrocodone is known chemically as 4,5α-Epoxy-3-methoxy-17-methylmorpinan-6-one.

Codeine is a centrally-acting narcotic, opiate analgesic. Its actions are qualitatively similar to morphine, but its potency is substantially less. Codeine is known chemically as 7,8-didehydro-4,5α-Epoxy-3-methoxy-17-methylmorpinan-6-α-ol.

Oxycodone is a semisynthetic pure agonist opioid whose principal therapeutic action is analgesia. Its actions are qualitatively similar to that of morphine; the most prominent involves the central nervous system and organs composed of smooth muscle. Oxycodone is known chemically as 4,5-Epoxy-14-hydroxy-3-methoxy-17-methylmorphinan-6-one.

Oxymorphone is a semisynthetic opioid analgesic. Administered parenterally, 1 mg of oxymorphone hydrochloride is approximately equivalent in analgesic activity to 10 mg of morphine sulfate. Oxymorphone is known chemically as 4,5α-Epoxy-3,14-dihydroxy-17-methylmorphinan-6-one.

Morphine is a natural product that is the prototype for the class of natural and synthetic opioid analgesics. Morphine produces both its therapeutic and its adverse effects by interaction with one or more classes of specific opioid receptors located throughout the body. Morphine acts as a pure agonist, binding with and activating opioid receptors at sites in the peri-aqueductal and peri-ventricular grey matter, the ventro-medial medulla and the spinal cord to produce analgesia. Morphine is known chemically as 7,8-Didehydro-4,5α-epoxy-17-methylmorphinan-3,6α-diol.

Hydromorphone is a hydrogenated ketone of morphine and is a narcotic analgesic. Its principal therapeutic effect is relief of pain. There is no intrinsic limit to the analgesic effect of hydromorphone; like morphine, adequate doses will relieve even the most severe pain. Hydromorphone is known chemically as 4,5α-Epoxy-3-hydroxy-17-methylmorphinan-6-one.

Tannate compositions are currently widely used only in the treatment of upper respiratory symptoms associated with respiratory tract conditions such as the common cold, bronchial asthma, acute and chronic bronchitis. Such tannate compositions consist of various combinations of active ingredients in the tannate form from the antihistamine, decongestant, expectorant, and or antitussive classes.

Tannate salts are typically prepared by reacting the drug free base with tannic acid in the presence of a volatile solvent, such as isopropanol, or water and then vacuum or freeze dried. Reaction variables such as mixing time and temperatures vary depending on the drug molecule and solvent used. Other methods of tannate preparation include the mixing of solid free base with solid tannic acid under heated conditions until completely converted to the tannate salt. Also, various in-situ methods are employed to convert compounds from their more commonly available salt forms such as citrate, hydrochloride, or maleate to the tannate salt as a part of the dosage form processing.

A considerable number of tannic acids occur in nature. Chemically, these acids are described as polymers of different hydroxybenzoic acids. Generally, when the term tannic acid is employed, as in the present case, the acid referred to is gallotannic acid. The internal ester of gallic acid also frequently referred to as tannin.

Tannic acid consists of an amorphous powder, glistening scales, or spongy masses varying in color from yellowish-white to light brown. Tannic acid is very soluble in water or alcohol.

Commercially available, tannic acid, also known as tannin, has a complex non-uniform chemistry, usually contains from about 5% to about 10% water by weight, has a molecular weight of about 1700, and is typically produced from Turkish or Chinese nutgall.

### DETAILED DESCRIPTION OF THE INVENTION

It has now been found that the novel use of opiate and non-opiate tannate compounds such as but not limited to hydrocodone tannate, codeine tannate, oxycodone tannate, oxymorphone tannate, morphine tannate, and hydromorphone tannate, alone or in combination with one or more additional active ingredients and novel combinations containing these tannates produce a composition possessing analgesic properties.

The present invention is directed to methods and compositions for treating pain in humans and animals, both adult and juvenile, comprising administration of compositions comprising analgesic (opiate and/or non-opiate) tannates such as but not limited to hydrocodone tannate, codeine tannate, oxycodone tannate, oxymorphone tannate, morphine tannate, and hydromorphone tannate, alone or in combination with one or more additional active ingredients. Such additional active ingredients may include tannate compounds and/or non-tannate compounds.

It is believed that tannate salts of active agents provide therapeutic activity for longer time periods. In effect, the inclusion of an active agent in a tannate salt form extends the release profile of the active agent and there is less spiking in pharmacological effect of the active agent. This leads to better compliance by the patient in that the active agent in the tannate salt form does not need to be given as often and there are fewer side effects, particularly from over dosage effects. This extended release profile has been demonstrated in-vitro. In preferred embodiments, the release profile of the active analgesic tannate is increased relative to the non-tannate form of the analgesic by at least 50%, 100%, 200%, 300%, 400%, 500%, 600%, 700%, 800%, 900% or 1000%. In some preferred embodiments, the release profile is increased relative to the non-tannate form of the analgesic by at least 500%.

The tannate compositions of the present invention can be made by methods known to those skilled in the art. Preparations of tannate compounds in a very pure form are taught in U.S. Pat. Nos. 5,599,846 and 5,663,415 to Chopdekar et al., which are herein incorporated in their entireties. In general, one method of making tannate compounds comprises reacting the base compound, such as chlorpheniramine or brompheniramine, with tannic acid in a solvent such as alcohol.

The compositions described herein can be designed to be taken twice a day in order to utilize the prolonged analgesic action of hydrocodone tannate, codeine tannate, oxycodone tannate, oxymorphone tannate, morphine tannate, and/or hydromorphone tannate. The action of hydrocodone tannate, codeine tannate, oxycodone tannate, oxymorphone tannate, morphine tannate, and/or hydromorphone tannate may be utilized alone or in combination with the prolonged action of other compounds, either tannate or non-tannate in nature, or the immediate action of other compounds. The compositions of the present invention may be prepared for oral administration in the form of powders, capsules, elixirs, syrups, suppositories and the preferred forms of tablets or suspensions. Administration by any other known route is also contemplated, such as transmucosally, transdermally, intravenously, intramuscularly or intraparenterally.

Tablets containing the unique hydrocodone tannate, codeine tannate, oxycodone tannate, oxymorphone tannate, morphine tannate, and/or hydromorphone tannate compositions of the present invention are prepared in a conventional manner by the addition of suitable pharmaceutical carriers including fillers, diluents, colorants, lubricants and the like, as well as conventional and well known binding and disintegrating agents. Chewable tablet formulations would also include ingredients to enhance flavor and palatability such as sweeteners and natural and artificial flavors. Each tablet comprises, or consists essentially of, approximately 1 to 60 mg of hydrocodone tannate, approximately 1 to 60 mg. of hydrocodone tannate, about 1 to 240 mg of codeine tannate, about 1 to 400 mg of oxycodone tannate, about 1 to 60 mg of oxymorphone tannate, about 1 to 300 mg of morphine tannate, and/or about 1 to 60 mg of hydromorphone tannate alone. The compositions may also comprise a therapeutic amount of another pharmaceutically active ingredient. A typical chewable tablet composition of the present invention containing compressible sugar, magnesium stearate, microcrystalline cellulose (Avicel CE-15), citric acid, and flavor as described in Example 1 which follows, is prepared by well-known conventional tabletting techniques such as those disclosed in U.S. Pat. Nos. 3,018,221; 2,798,024 and 2,757,124.

### EXAMPLE 1

Hydrocodone Tannate Tablets

| Ingredient | Milligrams per Tablet |
|---|---|
| Hydrocodone tannate | 20.00 |
| Compressible sugar, NF | 247.40 |
| Microcrystalline cellulose, NF | 30.00 |
| Citric acid, USP | 0.20 |
| Berry flavor | 0.90 |
| Magnesium Stearate, NF | 1.50 |

Tablets containing combinations of hydrocodone tannate and one or more additional active ingredients would comprise essentially the same ingredients in the same amounts. Changes in the additional active ingredient(s) present would be offset by the appropriate addition or subtraction to the compressible sugar amount. Total tablet weight would remain the same.

### EXAMPLE 2

Codeine Tannate Tablets

| Ingredient | Milligrams per Tablet |
|---|---|
| Codeine tannate | 120.00 |
| Compressible sugar, NF | 147.30 |
| Microcrystalline cellulose, NF | 30.00 |
| Citric acid, USP | 0.30 |
| Berry flavor | 0.90 |
| Magnesium Stearate, NF | 1.50 |

Tablets containing a combination of codeine tannate and one or more additional active ingredients would comprise essentially the same ingredients in the same amounts with the exception of the additional active ingredient(s) in place of the same amount by weight of compressible sugar.

### EXAMPLE 3

Oxycodone Tannate Tablets

| Ingredient | Milligrams per Tablet |
|---|---|
| Oxycodone tannate | 20.00 |
| Compressible sugar, NF | 247.40 |
| Microcrystalline cellulose, NF | 30.00 |
| Citric acid, USP | 0.30 |
| Berry flavor | 0.90 |
| Magnesium Stearate, NF | 1.50 |

Tablets containing a combination of oxycodone tannate and one or more additional active ingredients would comprise essentially the same ingredients in the same amounts with the exception of the additional active ingredient(s) in place of the same amount by weight of compressible sugar.

### EXAMPLE 4

Oxymorphone Tannate Tablets

| Ingredient | Milligrams per Tablet |
|---|---|
| Oxymorphone tannate | 20.00 |
| Compressible sugar, NF | 247.40 |
| Microcrystalline cellulose, NF | 30.00 |
| Citric acid, USP | 0.20 |
| Berry flavor | 0.90 |
| Magnesium Stearate, NF | 1.50 |

Tablets containing a combination of oxymorphone tannate and one or more additional active ingredients would comprise essentially the same ingredients in the same amounts with the exception of the additional active ingredient(s) in place of the same amount by weight of compressible sugar.

### EXAMPLE 5

Morphine Tannate Tannate Tablets

| Ingredient | Milligrams per Tablet |
|---|---|
| Morphine tannate | 20.00 |
| Compressible sugar, NF | 207.40 |
| Microcrystalline cellulose, NF | 30.00 |
| Citric acid, USP | 0.30 |
| Berry flavor | 0.90 |
| Magnesium Stearate, NF | 1.50 |

Tablets containing a combination of morphine tannate and one or more additional active ingredients would comprise essentially the same ingredients in the same amounts with the exception of the additional active ingredient(s) in place of the same amount by weight of compressible sugar.

### EXAMPLE 6

Hydromorphone Tannate Tablets

| Ingredient | Milligrams per Tablet |
|---|---|
| Hydromorphone tannate | 20.00 |
| Compressible sugar, NF | 247.40 |
| Microcrystalline cellulose, NF | 30.00 |
| Citric acid, USP | 0.30 |
| Berry flavor | 0.90 |
| Magnesium Stearate, NF | 1.50 |

Tablets containing a combination of hydromorphone tannate and one or more additional active ingredients would comprise essentially the same ingredients in the same amounts with the exception of the additional active ingredient(s) in place of the same amount by weight of compressible sugar.

Suspensions of the compositions of the present invention are prepared in a conventional manner such that each 5 mL (one teaspoon) would contain approximately 1 to 60 mg. of hydrocodone tannate, about 1 to 240 mg of codeine tannate, about 1 to 400 mg of oxycodone tannate, about 1 to 60 mg of oxymorphone tannate, about 1 to 300 mg of morphine tannate, and/or about 1 to 60 mg of hydromorphone tannate alone or in combination with a therapeutic amount of another pharmaceutical active ingredient. Additionally, the suspension formulations may contain additional ingredients such as, but not limited to, citric acid, colorants, natural and artificial flavors, glycerin, magnesium aluminum silicate, methylparaben, propylparaben, purified water, sodium citrate, sweeteners such as sucralose, sucrose, or sorbitol, and xanthan gum. Example 7, which follows, is illustrative of a typical suspension formulation of the present invention prepared by conventional well-known compounding techniques.

### EXAMPLE 7

Hydrocodone Tannate Suspension

| Ingredient | Milligrams per 5 mL |
|---|---|
| Hydrocodone tannate | 20.00 |
| Xanthan Gum, NF | 30.00 |
| Magnesium Aluminum Silicate, | 35.00 |
| NF | |
| Methylparaben, NF | 7.50 |
| Propylparaben, NF | 1.50 |
| Sucralose, NF | 7.50 |
| Glycerin, USP | 250.00 |
| Citric Acid, USP | 10.00* |
| Sodium Citrate, USP | 2.50* |
| Artificial Berry Flavor | 15.00 |
| FD&C Red #40 Dye | 0.20 |
| Purified Water, USP (Deionized) | Adjust to 5 mL |

*Additional Citric Acid or Sodium Citrate may also be included in the formula if needed for pH adjustment.

Suspensions containing combinations of hydrocodone tannate and one or more additional active ingredients would comprise essentially the same ingredients in the same amounts. Changes in the additional active ingredient(s) present would be offset by the appropriate addition or subtraction to the purified water content.

### EXAMPLE 8

Codeine Tannate Suspension

| Ingredient | Milligrams per 5 mL |
|---|---|
| Codeine tannate | 120.00 |
| Xanthan Gum, NF | 30.00 |
| Magnesium Aluminum Silicate, | 35.00 |
| NF | |
| Methylparaben, NF | 7.50 |
| Propylparaben, NF | 1.50 |
| Sucralose, NF | 7.50 |
| Glycerin, USP | 250.00 |
| Citric Acid, USP | 10.00* |
| Sodium Citrate, USP | 2.50* |
| Artificial Berry Flavor | 15.00 |
| FD&C Red #40 Dye | 0.20 |
| Purified Water, USP (Deionized) | Adjust to 5 mL |

*Additional Citric Acid or Sodium Citrate may also be included in the formula if needed for pH adjustment.

Suspensions containing a combination of codeine tannate and one or more additional active ingredients would comprise essentially the same ingredients in the same amounts with the exception of the additional active ingredient(s) in place of the same amount by weight of purified water.

### EXAMPLE 9

Oxycodone Tannate Suspension

| Ingredient | Milligrams per 5 mL |
|---|---|
| Oxycodone tannate | 20.00 |
| Xanthan Gum, NF | 30.00 |
| Magnesium Aluminum Silicate, | 35.00 |
| NF | |
| Methylparaben, NF | 7.50 |
| Propylparaben, NF | 1.50 |
| Sucralose, NF | 7.50 |
| Glycerin, USP | 250.00 |
| Citric Acid, USP | 10.00* |
| Sodium Citrate, USP | 2.50* |
| Artificial Berry Flavor | 15.00 |
| FD&C Red #40 Dye | 0.20 |
| Purified Water, USP (Deionized) | Adjust to 5 mL |

*Additional Citric Acid or Sodium Citrate may also be included in the formula if needed for pH adjustment.

Suspensions containing a combination of oxycodone tannate and one or more additional active ingredients would comprise essentially the same ingredients in the same amounts with the exception of the additional active ingredient(s) in place of the same amount by weight of purified water.

### EXAMPLE 10

Oxymorphone Tannate Suspension

| Ingredient | Milligrams per 5 mL |
|---|---|
| Oxymorphone tannate | 20.00 |
| Xanthan Gum, NF | 30.00 |
| Magnesium Aluminum Silicate, | 35.00 |
| NF | |
| Methylparaben, NF | 7.50 |
| Propylparaben, NF | 1.50 |
| Sucralose, NF | 7.50 |
| Glycerin, USP | 250.00 |
| Citric Acid, USP | 10.00* |
| Sodium Citrate, USP | 2.50* |
| Artificial Berry Flavor | 15.00 |
| FD&C Red #40 Dye | 0.20 |
| Purified Water, USP (Deionized) | Adjust to 5 mL |

*Additional Citric Acid or Sodium Citrate may also be included in the formula if needed for pH adjustment.

Suspensions containing a combination of oxymorphone tannate and one or more additional active ingredients would comprise essentially the same ingredients in the same amounts with the exception of the additional active ingredient(s) in place of the same amount by weight of purified water.

### EXAMPLE 11

Morphine Tannate Suspension

| Ingredient | Milligrams per 5 mL |
|---|---|
| Morphine tannate | 60.00 |
| Xanthan Gum, NF | 30.00 |
| Magnesium Aluminum Silicate, | 35.00 |
| NF | |
| Methylparaben, NF | 7.50 |
| Propylparaben, NF | 1.50 |
| Sucralose, NF | 7.50 |
| Glycerin, USP | 250.00 |
| Citric Acid, USP | 10.00* |
| Sodium Citrate, USP | 2.50* |
| Artificial Berry Flavor | 15.00 |
| FD&C Red #40 Dye | 0.20 |
| Purified Water, USP (Deionized) | Adjust to 5 mL |

*Additional Citric Acid or Sodium Citrate may also be included in the formula if needed for pH adjustment.

Suspensions containing a combination of morphine tannate and one or more additional active ingredients would comprise essentially the same ingredients in the same amounts with the exception of the additional active ingredient(s) in place of the same amount by weight of purified water.

### EXAMPLE 12

Hydromorphone Tannate Suspension

| Ingredient | Milligrams per 5 mL |
|---|---|
| Hydromorphone tannate | 20.00 |
| Xanthan Gum, NF | 30.00 |
| Magnesium Aluminum Silicate, | 35.00 |
| NF | |
| Methylparaben, NF | 7.50 |
| Propylparaben, NF | 1.50 |
| Sucralose, NF | 7.50 |
| Glycerin, USP | 250.00 |
| Citric Acid, USP | 10.00* |
| Sodium Citrate, USP | 2.50* |
| Artificial Berry Flavor | 15.00 |
| FD&C Red #40 Dye | 0.20 |
| Purified Water, USP (Deionized) | Adjust to 5 mL |

*Additional Citric Acid or Sodium Citrate may also be included in the formula if needed for pH adjustment.

Suspensions containing a combination of hydromorphone tannate and one or more additional active ingredients would comprise essentially the same ingredients in the same amounts with the exception of the additional active ingredient(s) in place of the same amount by weight of purified water.

The dosage administered will be dependent on the mode of administration, the specific opiate tannate utilized, in addition to the age, health and weight of the recipient, kinds of concurrent treatment, if any, frequency of treatment and effect desired.

It should be understood that the above examples are illustrative of the exemplary modes only of the invention herein disclosed. Given the present disclosure, it is anticipated that numerous variations will occur to those skilled in the art. A latitude of modification, substitution and change is intended and in some instances, some features of the invention will be employed without a corresponding use of other features. Accordingly, it is intended that the spirit and scope of the invention disclosed herein should be limited only by the following claims.

1. A therapeutic composition for the treatment of pain comprising pharmaceutically effective amounts of an active ingredient, said active ingredient consisting essentially of an analgesic tannate selected from hydrocodone tannate, codeine tannate, oxycodone tannate, oxymorphone tannate, morphine tannate, hydromorphone tannate and combinations thereof.

2. The therapeutic composition of claim 1 in tablet form.

3. The therapeutic composition of claim 1 in suspension form.

4. A method for treating and relieving pain comprising orally administering to a warm-blooded animal in need of such treatment a therapeutic amount of a composition comprising an active ingredient, said active ingredient consisting essentially of an analgesic tannate selected from hydrocodone tannate, codeine tannate, oxycodone tannate, oxymorphone tannate, morphine tannate, hydromorphone tannate and combinations thereof.

5. The method of claim 4 wherein said composition is in tablet form.

6. The method of claim 4 wherein said composition is in suspension form.

7. The therapeutic composition of claim 2, wherein said tablet form contains about 1 to 60 mg. of hydrocodone tannate, about 1 to 240 mg of codeine tannate, about 1 to 400 mg of oxycodone tannate, about 1 to 60 mg of oxymorphone tannate, about 1 to 300 mg of morphine tannate, or about 1 to 60 mg of hydromorphone tannate, or combinations thereof.

8. The therapeutic composition of claim 2, wherein said tablet form contains about 20 mg. of hydrocodone tannate.

9. The therapeutic composition of claim 2, wherein said tablet form contains about 120 mg. of codeine tannate.

10. The therapeutic composition of claim 2, wherein said tablet form contains about 20 mg. of oxycodone tannate.

11. The therapeutic composition of claim 2, wherein said tablet form contains about 20 mg. of oxymorphone tannate.

12. The therapeutic composition of claim 2, wherein said tablet form contains about 60 mg. of morphine tannate.

13. The therapeutic composition of claim 2, wherein said tablet form contains about 20 mg. of hydromorphone tannate.

14. The therapeutic composition of claim 3, wherein said suspension form contains about 1 to 60 mg of hydrocodone tannate, about 1 to 240 mg of codeine tannate, about 1 to 400 mg of oxycodone tannate, about 1 to 60 mg of oxymorphone tannate, about 1 to 300 mg of morphine tannate, or about 1 to 60 mg of hydromorphone tannate alone or combinations thereof per 5 mL.

15. The therapeutic composition of claim 3, wherein said suspension form contains about 20 mg. of hydrocodone tannate per 5 mL.

16. The therapeutic composition of claim 3, wherein said suspension form contains about 120 mg. of codeine tannate per 5 mL.

17. The therapeutic composition of claim 3, wherein said suspension form contains about 20 mg. of oxycodone tannate per 5 mL.

18. The therapeutic composition of claim 3, wherein said suspension form contains about 20 mg. of oxymorphone tannate per 5 mL.

19. The therapeutic composition of claim 3, wherein said suspension form contains about 60 mg. of morphine tannate per 5 mL.

20. The therapeutic composition of claim 3, wherein said suspension form contains about 20 mg. of hydromorphone tannate per 5 mL.

21. The method of claim 5, wherein said tablet form contains about 1 to 60 mg. of hydrocodone tannate, about 1 to 240 mg of codeine tannate, about 1 to 400 mg of oxycodone tannate, about 1 to 60 mg of oxymorphone tannate, about 1 to 300 mg of morphine tannate, or about 1 to 60 mg of hydromorphone tannate or combinations thereof.

22. The method of claim 5, wherein said tablet form contains about 20 mg. of hydrocodone tannate.

23. The method of claim 5, wherein said tablet form contains about 120 mg. of codeine tannate.

24. The method of claim 5, wherein said tablet form contains about 20 mg. of oxycodone tannate.

25. The method of claim 5, wherein said tablet form contains about 20 mg. of oxymorphone tannate.

26. The method of claim 5, wherein said tablet form contains about 60 mg. of morphine tannate.

27. The method of claim 5, wherein said tablet form contains about 20 mg. of hydromorphone tannate.

28. The method of claim 6, wherein said suspension form contains about 1 to 60 mg. of hydrocodone tannate, about 1 to 240 mg of codeine tannate, about 1 to 400 mg of oxycodone tannate, about 1 to 60 mg of oxymorphone tannate, about 1 to 300 mg of morphine tannate, or about 1 to 60 mg of hydromorphone tannate alone or combinations thereof per 5 mL.

29. The method of claim 6, wherein said suspension form contains about 20 mg. of hydrocodone tannate per 5 mL.

30. The method of claim 6, wherein said suspension form contains about 120 mg. of codeine tannate per 5 mL.

31. The method of claim 6, wherein said suspension form contains about 20 mg. of oxycodone tannate per 5 mL.

32. The method of claim 6, wherein said suspension form contains about 20 mg. of oxymorphone tannate per 5 mL.

33. The method of claim 6, wherein said suspension form contains about 60 mg. of morphine tannate per 5 mL.

34. The method of claim 6, wherein said suspension form contains about 20 mg. of hydromorphone tannate per 5 mL.
